(19) United States
(12) Patent Application Publication
(10) Pub. No.: US 2012/0282199 A1
(43) Pub. Date: Nov. 8, 2012

(54) UV PROTECTING COMPOSITION AND APPLICATOR

(76) Inventors: George Pollack, Fair Lawn, NJ (US); Janine Pollack, New York, NY (US)

(21) Appl. No.: 13/464,241

(22) Filed: May 4, 2012

Related U.S. Application Data
(60) Provisional application No. 61/482,818, filed on May 5, 2011.

(51) Int. Cl. A61K 8/37; B05C 17/02; A61Q 17/04

(52) U.S. Cl. 424/60; 401/208

(57) ABSTRACT

A composition that imparts protection from ultraviolet light to the skin of a subject is provided. The composition is suitable for delivery by a roller delivery apparatus that can be used to deliver the composition to areas of the body that are traditionally difficult to reach providing more complete and even coverage and enhancing the overall effect of protection from sunlight or other ultraviolet light.

### CROSS REFERENCE TO RELATED APPLICATIONS

This application depends from and claims priority to U.S. Provisional Application No. 61/482,818 filed May 5, 2011, the entire contents of which are incorporated herein by reference.

### FIELD OF THE INVENTION

The invention relates to protecting a subject from exposure to ultraviolet light. More particularly, the invention provides a photoprotective composition, applicator, and method of use.

### BACKGROUND OF THE INVENTION

Tanning of the human epidermis is a physiological result of exposure to light with wavelengths from 280 nm to 400 nm, representing the near ultraviolet (UV) spectrum. Exposure to light emissions in the wavelength range from 280 to 320 nm, known as UV-B rays, cause skin burns and erythema that not only lead to long term consequences such as development of cancerous growths, but also harm the desired development of a natural tan.

UV-A rays with wavelengths from 320 to 400 nm, promote tanning of the skin, but also cause unwanted damage. Continued or excessive exposure to UV-A light promotes reduced skin elasticity and the appearance of wrinkles. Thus, for health and aesthetic reasons such as the conservation of the skin's natural elasticity, it is increasingly important to control the effect of UV light exposure to the skin.

Typical sunscreens are formulated as a lotion to deliver UV absorbing agents to the skin that serve to absorb or otherwise filter UV light. Achieving even application of these sunscreen formulations is historically difficult leading to areas of heavy application adjacent to areas of light application. As such, some areas of the skin remain at risk for unwanted UV exposure. Recent efforts at improving ease of application led to the development of sunscreen compositions that could be delivered from a spray source. The application of these sunscreens still suffers from uneven application due to the difficulty in maintaining the spray source a constant distance from the skin and unevenness of the spray pattern. Moreover, spray delivery commonly requires sunscreen compositions that can be easily aerosolized. These typically are easily washed away requiring more frequent application to maintain an effective UV barrier.

Thus, there is a need for an improved sunscreen composition and delivery system.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A photoprotective composition is provided with specific viscosity, lubricity, and capability to be deliverable by a roller delivery apparatus. A photoprotective composition surprisingly achieves specific viscosity levels by the use of phenylethyl benzoate alone or in the presence of a low amount of triethanolamine. The photoprotective composition includes an emulsifier, phenylethyl benzoate at or less than 10% by weight, at least one UV-screening agent, and water as a predominant. In embodiments with triethanolamine, the triethanolamine is present at less than 0.1% by weight, optionally from 0.02 to 0.07% by weight.

A photoprotective composition is used alone or as part of a system. A system includes a delivery apparatus that has a dispensing head connected, optionally removably, to a reservoir and a roller housed rotatably associated with the dispensing head. The delivery apparatus contains or is coated at least in part with a photoprotective composition. The photoprotective composition is either housed within the reservoir, coated or impregnating the roller or both.

The photoprotective composition and system are optionally used in a method for protecting the skin of a subject from damage due to exposure to UV light.

### BRIEF DESCRIPTION OF THE DRAWINGS

FIG. 1 illustrates one embodiment of a delivery apparatus for delivery of a photoprotective composition;

FIG. 2 illustrates one embodiment of a roller operable for delivery of a photoprotective composition; and

FIG. 3 is a UV profile illustrating the stability of one embodiment of a photoprotective composition at 45° C. for 60 days.

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The invention has utility as a photoprotective composition. Among the vast array of possible delivery systems, the inventors discovered that a modified pump-roller system provided the greatest ability to deliver a photoprotective composition to traditionally difficult to reach areas as well as created an even application thereby promoting consistent protection from unwanted damage from UV exposure or for improved natural tanning of the skin. An example of a delivery apparatus is described in U.S. Pat. Nos. 6,244,776 and 6,126,352.

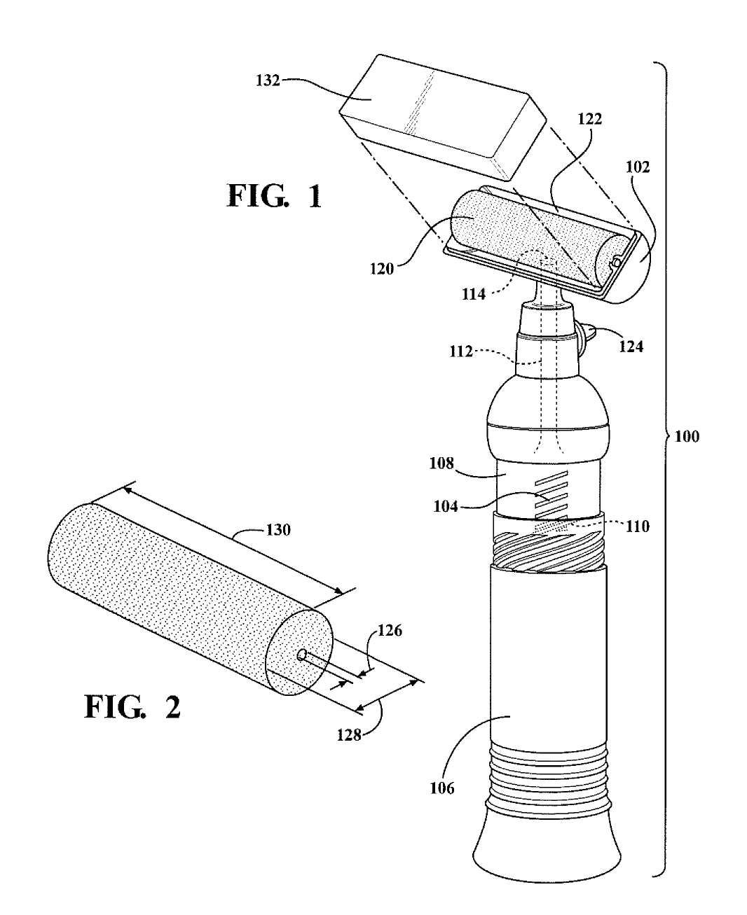

One embodiment of a delivery apparatus suitable for delivery of a photoprotective composition is illustrated in FIG. 1. A delivery apparatus typically includes a dispenser 102 mounted in association with a reservoir 104 suitable for housing an inventive photoprotective composition. The reservoir 104 optionally includes a telescoping extender 106. An extender optionally defines a portion of the reservoir allowing for adjustment of reservoir volume or promoting movement of a photoprotective composition toward the dispenser. Optionally, a reservoir is self contained and the extender serves to lengthen or shorten the delivery apparatus to promote reaching areas distant from a user's hand or for compacting the system for storage.

The reservoir 104 has an outer body surface 108, optionally in a cylindrical shape, that defines an axis carrying screw threads used for telescoping the extender 106. An extender 106 is optionally a cylindrical tube with an interior diameter sized to fit over the outer body surface 108 of the reservoir 104. The interior of the telescoping extender optionally includes screw threads that are complementary to and engage screw threads on the body surface. In exemplary embodiments, rotating the telescopic extender around its central cylindrical axis promotes extension or retraction of the extender relative to the body thereby adjusting the volume of the reservoir or simply extending or retracting the extender.

In the closed, upper position, the reservoir-extender are short for packaging and storage. When the extender is screwed out, the entire structure is lengthened so that when manually held, the dispensing head can reach typically difficult to reach parts of the body such as the center of the user's back. The telescoping extender can be extended downward to a stop 110 optionally formed by stop faces in both of the thread surfaces. The stop 110 is engaged when the extender is extended to its maximum practical length. From the maximum extended position, the extender can optionally be screwed upwardly to the point where the bottom of the extender is even with the bottom of the reservoir. The axis and rotation of the extender corresponds to the central axis of the reservoir to define the general length orientation of the reservoir with its extender which is its axis.

The reservoir 104 includes a mating system for association with a dispensing head. A mating system is optionally a screw-threaded nozzle onto which the dispensing head 102 is rotatably or fixedly attached. The dispensing head 102 has corresponding screw threads on its interior so that it may be attached and detached from the reservoir, as required.

A dispensing head 102 has an interior passage 112 that fluidly connects to a reservoir 104 at a first end and terminates in dispensing opening 114 at a second end that is fluidically associated with a roller 120. The surprisingly functional and stable viscosity of a photoprotective composition is operable with several diameters or cross sectional dimensions of a first opening as the viscosity and lubricity of the composition maintain its positioning within the interior passage independent of the size of the first end opening. A dispensing roller 120 fits into a pocket 122 formed by a basket in the dispensing head by associating with cradles to retain the roller in place. Cradles are optionally in the form of short slots parallel to walls of a basket so that the dispensing roller can move toward and away from the dispensing opening or are shaped so as to rotatably hold a roller in place. The roller 120 can optionally move sufficiently away from the dispensing opening 114 to permit dispensing of the fluid material in the reservoir 104 onto the underside of the roller 120. Upon moving the roller across the skin surface, the roller rolls dispensing the photoprotective composition on to the desired surface.

A delivery apparatus 100 optionally includes a dispensing button 124 similar to pressure devices found in airless pumps. A dispensing button 124 is fluidically associated with the interior passage 112 whereby pressing the button 124 into a collapsed configuration promotes movement of a photoprotective composition toward the roller 120. Releasing the button 124 promotes drawing photoprotective composition from the reservoir into the interior passage so that the system is primed for delivery of additional photoprotective composition onto the roller. A cover 132 is optionally included that removably attaches to the dispensing head 102 to cover the roller 120 thereby forming a substantially enclosed space suitable for storage or transport. A cover 132 is illustratively depicted as a rectangular shape, but such a cover optionally is semicircular or other shape to complement the shape of the roller 120. Other cover shapes are also operable.

In some embodiments, a reservoir 104 is formed of a flexible material that can be collapsed by the hand of a user to force a photoprotective composition from the reservoir toward the roller. This force is optionally a substitute for a dispensing button or is used as a complementary means of delivering photoprotective composition to the roller. As such, some embodiments include a squeezable reservoir 104, a dispensing button, or both to move photoprotective composition from the reservoir 104 onto the roller.

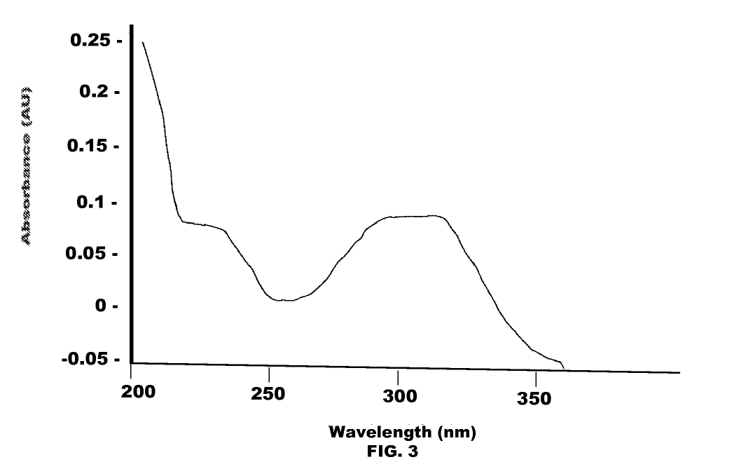

The roller in one embodiment is illustratively depicted in FIG. 2. Prior delivery apparatuses are incapable of effectively delivering a photoprotective composition. In some embodiments, particular relationships exist between the outer dimensions of the roller, the dispensing head, and the viscosity of a photoprotective composition so that the composition can be readily applied to the skin. Optionally, the roller is dimensioned to allow swelling to an outer diameter of the outer surface that falls within 2-5 mm from the outlet of the interior passage in the dispensing head. Optionally, a roller need not swell to a particular outer dimension; illustratively but not limited to situations when the viscosity of the photoprotective composition is ideal for transfer onto a roller absent roller swelling. Optionally, the length of the roller is such that it is at least 0.5 mm from the edge of the dispensing head when the roller is swelled. These dimensions are optionally used to allow photoprotective composition with suitable viscosity to be dispensed onto the roller both prior to the roller swelling with photoprotective composition and after the roller has swelled. The viscosity of the photoprotective composition both allows photoprotective composition to be effectively dispensed onto the roller and prevents it from dripping out of the delivery apparatus. In some embodiments, the viscosity of the photoprotective composition will coat all or a portion of the roller prior to any roller swelling. In some embodiments, an inventive dispenser includes a roller with dimensions inner diameter 126 of about 6 mm, an outer diameter 128 of about 22 mm to 46 mm, optionally 30 to 40 mm, and a length 130 of about 60 to 85 mm, optionally 73 mm, with the term “about” used herein defining dimensions within 10% of the exemplary dimension values, and where each of these values represents sponge dimensions prior to contact with photoprotective composition. The roller may, but is not necessarily required to in all embodiments, expand by 10-20% in outer radius and expand by 10-15% in length upon saturation with photoprotective composition.

A roller is optionally formed from any suitable material that is resistant to breakdown by any component of a photoprotective composition. Illustrative roller nap fiber materials include: polymeric materials such as polyesters illustratively polyethylene terephthalate, polyurethane, and polyacrylonitrile; natural materials such as wool; and combinations of these or other materials. A roller core is optionally a traditional core known in the art such as a core formed from paper illustratively a cardboard core, or is a solvent resistive material such as phenolic materials among others known in the art. A roller nap fiber is associated with a core via a solvent resistant adhesive. Illustrative examples of adhesives include those based on epoxy or others known in the art. Illustrative embodiments of a roller can be obtained from Sun Laboratories, Chatsworth, Calif.

Existing sunscreen formulations were incapable of being delivered by a delivery apparatus system as described herein or in U.S. Pat. Nos. 6,244,776 and 6,126,352 due to clogging of the pump apparatus or tendency to flow from the site of application. The inventors, therefore, determined that materials of a limited viscosity range and lubricity could be effectively delivered by a delivery apparatus. Of equal importance was formulating a composition capable of maintaining a target viscosity for a period of time to provide a useable lifetime to the product. The knowledge in the art suggested that formulating a photoprotective composition with a low level of emulsifier should achieve both these outcomes. Initial attempts failed to produce the desired viscosity range with the exception of a phosphate emulsifier containing cetearyl alcohol, dicetyl phosphate, and ceteth-10 phosphate. While this formulation provided a desired initial viscosity of 2000 cps, and a settled viscosity of 3800 cps, trial applications resulted in a clogged delivery apparatus and an unusable product. Subsequent adjusting of the phosphate emulsifier reduced the viscosity, but still resulted in clogging of the delivery apparatus. Subsequent addition of lubricating materials such as silicones or mineral oil similarly failed to improve the composition.

Unexpectedly, the inventors discovered that phenylethyl benzoate present in the composition in the narrow concentration range of 3% to 7% by weight alone or in the presence of triethanolamine at 0.02% to 0.07% by weight provided the necessary viscosity and lubricity to the final composition. As such, an inventive photoprotective composition includes exactly or less than 10% phenylethyl benzoate by weight, optionally from 3% to 7% or any value or range of phenylethyl benzoate by weight therebetween. Particularly preferred embodiments also include 0.02% to 0.07%, preferably about or precisely 0.05% by weight, triethanolamine as a viscosity adjusting neutralizer. These use of phenylethyl benzoate at 5% by weight in the presence of triethanolamine at 0.05% by weight provided the most beneficial formulation viscosity when used with an applicator such as that described herein or in U.S. Pat. Nos. 6,244,776 and 6,126,352.

A photoprotective composition includes one or more UV-screening agents, phenylethyl benzoate at or less than 10% by weight, an emulsifier, and water as a predominant.

Illustrative examples of UV-screening agents illustratively include avobenzone, benzophenone-3, p-aminobenzoic acid (PABA), camphor benzalkonium methosulfate, homosalate, phenylbenzimidazole sulfonic acid, terephthalidene dicamphor sulfonic acid, benzylidene camphor sulfonic acid, octocrylene, polyacrylamidomethyl benzylidene camphor, ethylhexyl methoxycinnamate, octylmethoxy cinnamate MCX, octyl salicylate, octyl crylene, PEG-25 PABA, isoamyl p-methoxycinnamate, ethylhexyl triazone, drometrizole trisiloxane, diethylhexyl butamido triazone, 4-methylbenzylidene camphor, 3-benzylidene camphor, ethylhexyl salicylate, ethylhexyl dimethyl PABA, benzophenone-4, benzophenone-5, methylene bis-benztriazolyl tetramethylbutylphenol, disodium phenyl dibenzimidazole tetrasulfonate, bis-ethylhexyloxyphenol methoxyphenol triazine, polysilicone-15, among others known in the art, as well as combinations thereof.

A UV-screening agent optionally filters UV-A light, UV-B light, or combinations thereof. A photoprotective composition includes from 0.001% to 10% by weight of any singular UV-screening agent. Optionally, a UV-screening agent is present at concentrations from 1% to 20% by weight or any value or range therebetween. Optionally, a UV-screening agent is present from 2% to 15% by weight. Optionally, a UV-screening agent is present from 3% to 7.5% by weight. One of skill in the art readily understands the amount of UV screening agent necessary to produce a desired sun protection factor. A photoprotective composition optionally includes 1, 2, 3, 4, 5, 6, 7, 8, or more UV-screening agents. In some embodiments, 2 or more UV-screening agents are provided where two or more of the agents work in concert such as illustratively described in U.S. Pat. No. 6,627,180. Optionally, two or more UV-screening agents have overlapping absorption spectra. UV-screening agents may be obtained from commercial suppliers, illustratively International Specialty Products, Wayne, N.J.

A photoprotective composition optionally includes one or more emulsifiers. An emulsifier is optionally included to enhance emulsification of one or more UV-screening agents or other component of a photoprotective composition. An example of a type of emulsifier is a surfactant. Illustrative examples of emulsifiers include proteins such as soy or other lechtins, dimethicone copolyol, propylene glycol stearate, sorbitan tristearate, sorbitan monooleate, sorbitan trioleate, emulsifying wax, cetearyl alcohol, polysorbate 20, and ceteareth 20, among others known in the art. Some embodiments include a phosphate emulsifier. A phosphate emulsifier optionally is cetearyl alcohol, dicetyl phosphate and ceteth-10 phosphate such as the emulsifier CRODAFOS CES sold by Croda, Inc., East Yorkshire, UK.

A photoprotective composition includes phenylethyl benzoate such as that sold under the tradename XTEND 226 by International Specialty Products, Wayne, N.J. Phenylethyl benzoate is present at or less than 10% by weight, optionally between 0.01% and 10% by weight or any value or range therebetween. Optionally, phenylethyl benzoate is present from 3% to 7% by weight or any value or range therebetween. Optionally, phenylethyl benzoate is present at 5% by weight. In some embodiments, phenylethyl benzoate is intermixed with one or more UV-screening agents prior to combining the UV-screening agents with water in a water-predominant photoprotective composition.

A photoprotective composition includes water as a predominant. In some embodiments water is present at greater than 55%, 60%, 65%, 70%, or 75% by weight. Optionally, water is present from 55% to 70% by weight. Optionally, water is present from 60% to 70% by weight. Water is optionally purified to remove contaminants such as solids and other microorganisms, or subjected to processes to remove contaminating ions. Illustratively, compositions include deionized water prepared by methods and using apparatuses known in the art. Methods of purifying or filtering water are well known in the art.

A photoprotective composition optionally includes one or more additives. An additive is optionally: a lubricant; preservative; thickener; solvent; skin conditioner such as a stearic acid illustratively that sold under the tradename CRODAMOL OS sold by Croda, Inc. Edison, N.J.; humectant; color; fragrance; antioxidant; anti-microbial agent such as a mixture of propylene glycol, propylparaben, methylparaben, and diazolidinyl urea typically sold under the tradename GERMABEN sold by International Specialty Products, Wayne, N.J.; natural extract; vitamin such as vitamin E, vitamin A, vitamin D, vitamin C or combinations thereof; opacifying agent; styling polymer; anti-static agent; pediculocide; or combinations thereof. The levels of an additive are readily determinable to one of ordinary skill in the art.

In some embodiments, an additive is a natural extract. Illustrative examples of natural extracts include extracts of aloe vera, ginseng, cucumber, chamonille, or other extracts known in the art. Natural extracts are optionally available from Active Organics, Lewisville, Tex. Natural extracts are optionally present at levels readily determinable by those of skill in the art, optionally at or less than 0.5% by weight, optionally at 0.1% by weight, optionally between 0.001 and 2% by weight or any value or range therebetween.

A photoprotective composition has a viscosity upon final formulation in the range of 6000 to 7500 cps. It is appreciated that the viscosity is altered during storage. The viscosity is optionally adjusted 24 hours after initial formulation and prior to loading a photoprotective composition into a dispensing apparatus to form a system for applying a photoprotective composition. A viscosity adjusting neutralizer is used to adjust the viscosity to a desired level optionally such that photoprotective composition has a final viscosity of 9000 cps to 12500 cps. This viscosity is stable for the life of the product. A viscosity adjusting neutralizer is illustratively triethanolamine. In particularly preferred embodiments, triethanolamine is present at from 0.02% to 0.07% by weight. Exceptional results are obtained with 0.05% triethanolamine.

A photoprotective composition is optionally stable (no appreciable breakdown) for a period of 60 days or more at 45° C.

A photoprotective composition is optionally used alone or packaged into a delivery apparatus for application to the skin of a subject. The combination of a photoprotective composition and a delivery apparatus each as described herein forms a system. A system is useful for delivering a photoprotective composition to the skin of a subject. As used herein the term “subject” is illustratively a mammal. Illustrative examples of a subject include a human or non-human primate, equine, bovine, murine, and a pig. It is appreciated that a system incorporates any photoprotective composition herein. Particularly preferred systems incorporate a composition with between 0.001% and 10% by weight phenylethyl benzoate and 0.02% to 0.07% by weight triethanolamine. Exceptional results are achieved by combining a photoactive composition including between 5% by weight phenylethyl benzoate and 0.05% by weight triethanolamine

A method of protecting the skin from exposure to UV light or preventing undesirable consequences of UV exposure to the skin of a subject includes applying a photoprotective composition to the skin of a subject. A photoprotective composition is optionally applied using a delivery apparatus substantially as described herein. A photoprotective composition is optionally applied once daily, twice daily, or at other desirable intervals. Optionally, a photoprotective composition is applied every one, two, three, four, five, or six hours. Optionally, a photoprotective composition is applied following submersion in water. The skin of a subject is protected from damage resulting from UV exposure by the step of applying the photoprotective composition. Applying is by any suitable method to contact a photoprotective composition with the skin of a subject. Illustratively, a photoprotective composition is applied by contacting a roller of a delivery apparatus to the skin of the subject whereby the roller contains a photoprotective composition and moving the roller along the surface of the skin to dispense photoprotective composition onto the skin. Experimental trials demonstrate effective application of a photoprotective composition to the skin of a subject.

The photoprotective composition is optionally used to prevent undesirable consequences of UV exposure to the skin. Undesirable consequences illustratively include, but are not limited to skin burns, erythema, pre-cancerous (illustratively actinic keratosis) and cancerous (illustratively squamous cell carcinoma, basal cell carcinoma, and melanoma) skin lesions, benign tumors, fine and coarse wrinkles, freckles, mottled pigmentation, sallowness, telangiectasias, and elastosis. Illustrative undesirable consequences are illustrated by Green, et al., Prog Biophys Mol Biol. 2011; 107(3):349-55 and Narayanan, et al., Int J Dermatol, 2010; 49(9):978-86.

### EXAMPLES

### Example 1

### Photoprotective Compositions

A photoprotective composition is formulated by combining five phases to form a composition with suitable protective capacity, viscosity, and lubricity to be suitable for delivery by a delivery apparatus. Phase A is formed by the addition of a desired amount of deionized water and propylene glycol to a mixing kettle (by the addition of 10% excess water to account for any water loss, if necessary). A viscosity modifier is sprinkled into the water with mixing until well dispersed and no particles exist. The resulting gum is allowed to swell. The kettle is then gently heated to 75-80° C.

A Phase B is formed in a separate kettle and heated to 75-80° C. Phase B ingredients include an emulsifier, phenylethyl benzoate, UV screening agent(s), and optionally skin conditioner(s) and vitamins. When Phase B is melted, the entire contents are added to Phase A with thorough mixing to form an emulsion. The emulsion is mixed for a short period of time followed by cooling to 60° C. The cooled emulsion is then combined with propylene glycol, propylparaben, methylparaben, and diazolidinyl urea (GERMABEN II) as phase C followed by thorough mixing.

Phase D contains one or more natural extracts. Each extract is then added to the emulsion with thorough mixing. The anticipated weight and volume are checked and water is added if necessary. The composition is cooled and mixed until the temperature reaches 25° C. pH and viscosity are then determined and adjusted if required. The desired pH is 4.5-5.5.

Viscosity is measured using a RVT Brookfield Viscometer, #6 spindle, 20 rpm, 25° C. The viscosity of the system immediately following formation is between 500-2500 cps. The composition viscosity changes with time during an initial set up period. Within 24 hours after manufacturing the photoprotective composition has a viscosity of 3500-5000 cps. After 72 hours the viscosity is 6000-7500 cps. The final viscosity is adjusted by the addition of triethanolamine to reach a range of 9000-11500 cps, which is suitable for packaging and dispensing in a dispensing apparatus.

Illustrative compositions formed by the above procedure are highlighted in Formula A below.

Formula A:

| Ingredient | Percent (wt/wt) |
|---|---|
| Phase A | |
| Water (deionized) | q.s. to 100% |
| Viscosity modifier | 0.1 |
| Propylene Glycol | 3 |
| Phase B | |
| Emulsifier | 1.8 |
| Phenylethyl benzoate | 0.01-10 |
| UV screening agent(s) | 3-20 (each) |
| Skin conditioner | 1 |
| Vitamin | 0.5 |
| Phase C | |
| Antimicrobial | ≦1 |
| Phase D | |
| Natural extract(s) | 0.1 (each) |
| Phase E | |
| Triethanolamine | 0.02-0.07 |

Specific formulations of Formula A are formed with the indicated varied amounts of phenylethyl benzoate and triethanolamine as illustrated in Table 1 with all other components as listed in Formula A.

TABLE 1

| | Phenylethyl | Triethanolamine |
|---|---|---|
| Formula | Benzoate (% wt/wt) | (% wt/wt) |
| B | 1 | 0.05 |
| C | 2 | 0.05 |
| D | 3 | 0.05 |
| E | 4 | 0.05 |
| F | 5 | 0.05 |
| G | 6 | 0.05 |
| H | 7 | 0.05 |
| I | 8 | 0.05 |
| J | 9 | 0.05 |
| K | 10 | 0.05 |
| L | 1 | 0.02 |
| M | 2 | 0.02 |
| N | 3 | 0.02 |
| O | 4 | 0.02 |
| P | 5 | 0.02 |
| Q | 6 | 0.02 |
| R | 7 | 0.02 |
| S | 8 | 0.02 |
| T | 9 | 0.02 |
| U | 10 | 0.02 |
| V | 1 | 0.07 |
| W | 2 | 0.07 |
| X | 3 | 0.07 |
| Y | 4 | 0.07 |
| Z | 5 | 0.07 |
| AA | 6 | 0.07 |
| BB | 7 | 0.07 |
| CC | 8 | 0.07 |
| DD | 9 | 0.07 |
| EE | 10 | 0.07 |

One specific example of a photoprotective composition is illustrated in Table 2:

TABLE 2

| | | Percent |
|---|---|---|
| Ingredient | Trade Name | (wt/wt) |
| Phase A | | |
| Water (deionized) | | q.s. to |
| | | 100% |
| polyacrylate polymer | Carbopol 940 | 0.1 |
| Propylene Glycol | | 3 |
| Phase B | | |
| ceteth-10 phosphate | Crodafos CES | 1.8 |
| Phenylethyl benzoate | Xtend 226 (ISP) | 5 |
| Benzophenone-3 | Escalol 567 | 3 |
| Octylmethoxy cinnamate | Escalol 557 | 7.5 |
| MCX | | |
| Octyl salicylate | Escalol 587 | 5 |
| Octyl crylene | Escalol 597 | 5 |
| Octyl stearate | Crodamol OS | 1 |
| Tocopherol Acetate | Vitamin E | 0.5 |
| Phase C | | |
| Propylene Glycol, | Gemaben II | 1 |
| Methylparaben, | | |
| Propylparaben, | | |
| Diazolidinyl urea | | |
| Phase D | | |
| Aloe Vera Extract | Actiphyte of Aloe Vera Extract PG50 | 0.1 |
| Ginseng Extract | Actiphyte of Ginseng Extract PG50 | 0.1 |
| Cucumber Extract | Actiphyte of Cucumber Extract PG50 | 0.1 |
| Chamomile Extract | Actiphyte of Chamomile Extract | 0.1 |
| | PG50 | |
| Phase E | | |
| Triethanolamine | | 0.05 |

### Example 2

The photoprotective composition of Table 2 is subjected to stability testing by incubation in an oven at 25° C. or 45° C. for 60 days. UV spectra are obtained prior to and following incubation. The compositions of Table 2 and Formulas B-EE demonstrate excellent stability at both temperatures. FIG. 3 illustrates the UV spectra of the composition of Table 2 following incubation at 45° C. for 60 days.

### Example 3

The photoprotective compositions of Example 1 are packaged in the reservoir of a dispensing apparatus. The dispensing apparatus is purchased from Sun Laboratories, Chatsworth, Calif. and has unsaturated roller dimensions of inner diameter of 6 mm, outer diameter of 34 mm, and a length of 73 mm. The combination of the photoprotective composition and dispensing apparatus forms a system that allows application of the composition onto the skin of a subject efficiently and effectively.

### Example 4

The photoprotective composition is dispensed from the system onto the skin of a subject covering a skin area that is exposed to UV light. A control composition that is absent UV screening agent is applied from an identical dispensing apparatus over a similarly sized patch of skin. The test skin areas are exposed to light in the UVA range in a standard tanning bed for 10 minutes. The area with the photoprotective compositions of Example 1 demonstrate reduced redness and irritation.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

Patents and publications mentioned in the specification are indicative of the levels of those skilled in the art to which the invention pertains. These patents and publications are incorporated herein by reference to the same extent as if each individual application or publication was specifically and individually incorporated herein by reference for the entirety of their teaching.

1. A photoprotective composition comprising:
an emulsifier;
phenylethyl benzoate, said phenylethyl benzoate at 0.01 to 10 percent by weight;
at least one UV-screening agent; and
water as a predominant.

2. The photoprotective composition of claim 1 wherein said phenylethyl benzoate is present from 2 percent to 7 percent by weight.

3. The photoprotective composition of claim 1 wherein phenylethyl benzoate is present at 5 percent by weight.

4. The photoprotective composition of claim 1 further comprising triethanolamine at 0.02 to 0.07 percent by weight.

5. The photoprotective composition of claim 1 further comprising triethanolamine at 0.05 percent by weight

6. The photoprotective composition of claim 1 comprising from two to four UV-screening agents.

7. The photoprotective composition of claim 1 wherein said emulsifier comprises cetearyl alcohol, dicetyl phosphate and ceteth-10 phosphate.

8. The photoprotective composition of claim 1 further comprising an additive.

9. The photoprotective composition of claim 8 wherein said additive is a natural extract, a vitamin, an antimicrobial agent, or combinations thereof.

10. The photoprotective composition of claim 1 packaged into a reservoir.

11. The photoprotective composition of claim 1 further associated with a roller.

12. A system for applying a photoprotective composition to a surface comprising:
a delivery apparatus, said delivery apparatus comprising:
a dispensing head, said dispensing head connected to a reservoir; and
a roller housed rotatably associated with said dispensing head;
said delivery apparatus containing or coated at least in part with the photoprotective composition of claim 1, said composition within said reservoir, coating at least a portion of said roller, or both.

13. The system of claim 12 further comprising an extender movably connected to said reservoir.

14. The system of claim 12 wherein said photoprotective composition comprises phenylethyl benzoate present from 2 percent to 7 percent by weight.

15. The system of claim 12 wherein said photoprotective composition comprises phenylethyl benzoate present at 5 percent by weight.

16. The system of claim 12 wherein said photoprotective composition further comprises triethanolamine at 0.02 to 0.07 percent by weight.

17. The system of claim 15 wherein said photoprotective composition further comprises triethanolamine at 0.05 percent by weight.

18. A method of protecting skin from undesirable effects of UV exposure comprising:
applying the composition of claim 1 to the skin of a subject.

19. The method of claim 18 wherein said step of applying is using the system of claim 12.

20. The method of claim 18 wherein said step of applying is using the system of claim 17.
